(19) United States
(12) Patent Application Publication
(10) Pub. No.: US 2022/0282262 A1
(43) Pub. Date: Sep. 8, 2022

(54) SYNTHESIS OF THE FUCOSYLATED OLIGOSACCHARIDE LNFP-V

(71) Applicant: GLYCOM A/S, Hørsholm (DK)

(72) Inventors: Manos PAPADAKIS, Brønshøj (DK); Margit PEDERSEN, Roskilde (DK)

(21) Appl. No.: 17/299,583

(22) PCT Filed: Dec. 4, 2019
(86) PCT No.: PCT/IB2019/060423

§ 371 Date: Jun. 3, 2021

(30) Foreign Application Priority Data
Dec. 4, 2018 (DK) PA 2018 00952

(51) Int. Cl. C12N 15/70; C12N 9/10; C12P 19/18

(52) U.S. Cl. CPC C12N 15/70; C12N 9/1051; C12P 19/18; C12Y 204/01146; C12Y 204/01152; C12N 2330/51

(57) ABSTRACT

The present invention provides a method for biotechnological production of LNFP-V by using recombinant bacterial cells. LNFP-V is produced by using a polypeptide having an a1,3/4-fucosyl transferase activity and comprising or consisting of an amino acid sequence that has a sequence identity of at least 90% with amino acid sequence of SEQ ID No. 1 or 3.

### FIELD OF THE INVENTION

The present invention relates to the field of biotechnology, notably to a microbial production of the recombinant fucosylated oligosaccharide LNFP-V using a genetically modified microorganism, particularly E. coli, and the construction of said genetically modified cell.

### BACKGROUND OF THE INVENTION

In the present years, commercialization efforts for the synthesis of complex carbohydrates including oligosaccharides comprised in mammalian milk have increased significantly due to their roles in numerous biological processes occurring in living organisms. Human milk oligosaccharides (HMOs) are becoming important commercial targets for nutrition and therapeutic industries. More than 200 HMO species have now been reported and more than 130 HMO structures have been elucidated (Urashima et al.: Milk Oligosaccharides. Nova Biomedical Books, New York (2011); Chen Adv. Carbohydr. Chem. Biochem. 72, 113 (2015)). Although the synthesis and purification of HMOs with simpler structure, for example the trisaccharide 2′-fucosyllactose, in industrial scale, has recently been accomplished by multiple manufacturer using biotechnological methods comprising the utilization of genetically modified microorganisms, the same task for HMOs with more complicated structure is still challenging.

Lacto-N-fucopentaose V (LNFP-V) is a neutral pentasaccharide that was first isolated from human milk in 1976. Its structure was determined as tetrasaccharide lacto-N-tetraose (LNT) being fucosylated on the glucose residue with an α1,3-coupling (Galβ1-3GlcNAcβ1-3Galβ1-4(Fucα1-3)Glc, Scheme 1; Ginsburg et al. Arch. Biochem. Biophys. 175, 565 (1976)).

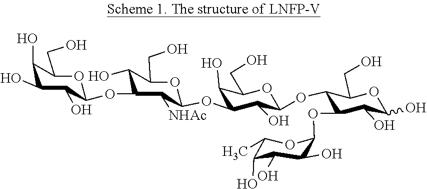

The average concentration of LNFP-V in human milk is 0.18 g/I (Erney et al. J. Pediatric GastroenteroL Nutr. 30, 181 (2000)). Due to its low concentration, the separation and isolation of LNFP-V from mother's milk does not seem to be economical. To date, no enzymatic or chemical total synthesis of LNFP-V has been reported. With regard to biotechnological methods, LNFP-V was produced in a lab-scale fermentation process from lactose using a recombinant E. coli comprising plasmid-borne heterologous genes lgtA, galTK and fucTIII encoding and expressing a β1,3-N-acetyl glucosaminyl transferase, a β1,3-galactosyl transferase and an α1,3/4-fucosyl transferase, respectively (M. Randriantsoa: Synthèse microbiologique des antigènes glucidiques des groupes sanguins, Thèse soutenue à I'Universitè Joseph Fourier, Grenoble, 2008).

Authors of Bioorg. Med. Chem. 23, 6799 (2015) and WO 2016/008602 disclosed a plasmid-free recombinant E. coli comprising the genes lgtA, wbgO and fucTIII encoding and expressing a β1,3-N-acetyl glucosaminyl transferase, a β1,3-galactosyl transferase and an α1,3/4-fucosyl transferase, respectively, which was, upon cultivation, able to produce, among others, the hexasaccharide LNDFH-II, but no formation of LNFP-V was reported.

Recently, it has been reported that LNFP-V showed a binding affinity to the carbohydrate binding domain of toxin A from Clostridium difficile (Nguyen et al. J. Microbiol. Biotechnol. 26, 659 (2016)). C. difficile is known to be the major cause of nosocomial diarrhoea (Kyne et al. Clin. Infect. Dis. 34, 346 (2002)).

Therefore, there is a need for a method that allows the production of sufficient amounts of isolated LNFP-V in a safe and cost-effective way.

### SUMMARY OF THE INVENTION

The present invention provides a method for biotechnological production of LNFP-V by using recombinant bacterial cells.

Accordingly, in a first aspect, the invention relates to a genetically modified microorganism or cell, preferably a bacterial cell, more preferably an E. coli cell, that comprises three functionally active heterologous glycosyl transferases selected from the group consisting of a β1,3-N-acetyl glucosaminyl transferase, a β1,3-galactosyl transferase and an α1,3/4-fucosyl transferase, wherein the α1,3/4-fucosyl transferase is encoded by a nucleic acid sequence selected from the group consisting of the fucT gene of H. pylori, the futA gene of H. pylori and functional variants/mutants thereof, and wherein the nucleic acid sequences encoding said heterologous glycosyl transferases are integrated in the genome of the microorganism or cell. Preferably, the recombinant microorganism or cell lacks intracellular β-galactosidase activity due to the deletion or inactivation of the native β-galactosidase gene, preferably lacZ.

A second aspect of the invention relates to method for producing LNFP-V, the method comprising:
providing a genetically modified microorganism or cell as disclosed in the first aspect of the invention,
culturing said microorganism or cell in the presence of lactose, and
separating LNFP-V from said microorganism or cell, from the culture medium or from both.

A third aspect of the invention relates to a protein (polypeptide) that comprises or consists of the amino acid sequence characterized by SEQ ID No. 7. The protein (polypeptide) that comprises or consists of the amino acid sequence characterized by SEQ ID No. 7 has α1,3/4-fucosyl transferase activity.

A fourth aspect of the invention relates to use of a polypeptide having an α1,3/4-fucosyl transferase activity in the production of LNFP-V, the polypeptide is selected from the group consisting of:
a polypeptide comprising or consisting of an amino acid sequence that has a sequence identity of at least 90% with amino acid sequence of SEQ ID No. 1, and
a polypeptide comprising or consisting of an amino acid sequence that has a sequence identity of at least 90% with amino acid sequence of SEQ ID No. 3.

### DESCRIPTION OF THE FIGURE

The invention will be described in further detail hereinafter with reference to the accompanying FIGURE, in which shows the alignment of H. pylori β1,3-galactosyl transferase sequence described in U.S. Pat. No. 6,974,687 (GenBank ID: BD182026) with the sequence of GalTK β1,3-galactosyl transferase encoded by galTK used in the examples of the present invention.

### DETAILED DESCRIPTION OF THE INVENTION

In the enzymatic synthesis of fucosylated lacto-N-tetraose (LNT), attention has been mostly focused on attaching fucose to N-acetylglucosamine, thereby constructing a structure carrying the Lewis A human antigen. The authors of Bioorg. Med. Chem. 23, 6799 (2015) and WO 2016/008602 constructed a genetically modified E. coli capable of synthesizing LNT and introduced a heterologous α1,3/4-fucosyl transferase in the cell (expressed from plasmid or genome integrated α1,3/4-fucosyl transferase fucTIII gene from H. pylori strain DSM 6709 (Rabbani et al. Glycobiology 15, 1076 (2005)). Upon cultivation, the main product detected and characterized was a double fucosylated LNT (lacto-N-difucohexaose II, LNDFH-II), bearing a first fucose residue on the N-acetylglucosamine and a second fucose moiety on the glucose. No monofucosylated LNT was identified among the products detected.

Other author (M. Randriantsoa: Synthèse microbiologique des antigènes glucidiques des groupes sanguins, Thèse soutenue à I'Universitè Joseph Fourier, Grenoble, 2008) demonstrated that a genetically modified E. coli strain that expresses a heterologous β1,3-N-acetyl glucosaminyl transferase, a heterologous β1,3-galactosyl transferase and the same heterologous α1,3/4-fucosyl transferase as mentioned above from plasmid was able to produce the monofucosylated lacto-N-tetraose LNFP-V, accompanied by LNT and LNDFH-II. Surprisingly, the present inventors were successful to construct a genome modified strain that produces high amounts of LNFP-V as main metabolic product by introducing selected heterologous genes encoding α1,3/4-fucosyl transferase.

Accordingly, the present invention relates to a genetically modified microorganism or cell, advantageously a bacterial cell, preferably E. coli, being capable of producing LNFP-V from lactose, and comprising:
a genome integrated heterologous nucleic acid sequence encoding a polypeptide having a β1,3-N-acetyl glucosaminyl transferase activity,
a genome integrated heterologous nucleic acid sequence encoding a polypeptide having a β1,3-galactosyl transferase activity, and
a genome integrated heterologous nucleic acid sequence encoding a polypeptide having an α1,3/4-fucosyl transferase activity,
   wherein the polypeptide having an α1,3/4-fucosyl transferase activity is selected from the group consisting of:
a polypeptide comprising or consisting of an amino acid sequence that has a sequence identity of at least 90% with amino acid sequence of SEQ ID No. 1, and
a polypeptide comprising or consisting of an amino acid sequence that has a sequence identity of at least 90% with amino acid sequence of SEQ ID No. 3.

Accordingly, the genetically modified microorganism or cell able to produce LNFP-V from lactose disclosed herein harbours and expresses three heterologous glycosyl transferase genes encoding proteins that are suitable and necessary for the synthesis of LNFP-V from lactose, namely a β1,3-N-acetyl glucosaminyl transferase, a β1,3-galactosyl transferase and an α1,3/4-fucosyl transferase, and said heterologous glycosyl transferase genes are integrated in the genome of the microorganism or cell. The heterologous α1,3/4-fucosyl transferase expressed is a polypeptide that comprises or consists of an amino acid sequence identical at least in 90% with SEQ ID No. 1 or SEQ ID No. 3.

In certain embodiments, the heterologous α1,3/4-fucosyl transferase expressed comprises or consists of a polypeptide that is identical with SEQ ID No. 1 or SEQ ID No. 3, whichever the case may be, in at least 92%, in at least 94%, in at least 95%, in at least 96%, in at least 97%, in at least 98% or in at least 99%.

The polypeptide of SEQ ID No. 1 is a truncated version of the native α1,3/4-fucosyl transferase of H. pylori NCTC 11639 (Gen Bank ID: AAB81031.1, Ge et al. J. Biol. Chem. 272, 21357 (1997), see SEQ ID No. 2 below), termed as “truncated FucT” herein. The truncated FucT lacks the 37 amino acids that constitute the C-terminus of the entire original protein characterized by SEQ ID No. 2 (Ma et al. J. Biol. Chem. 281, 6385 (2006)). The truncated FucT is encoded by the correspondingly truncated fucT gene of H. pylori NCTC 11639 (see GenBank ID: AF008596.1 for the entire fucT).

In one embodiment, the polypeptide comprising the amino acid sequence of SEQ ID No. 1 is the α1,3/4-fucosyl transferase of H. pylori NCTC 11639 (GenBank ID: AAB81031.1, Ge et al. J. Biol. Chem. 272, 21357 (1997)) in full length and characterized by SEQ ID No. 2, termed as FucT herein. FucT is encoded by the fucT gene of H. pylori NCTC 11639 (Gen Bank ID: AF008596.1).

The polypeptide of SEQ ID No. 3 is an α1,3/4-fucosyl transferase of H. pylori ATCC 26695 (GenBank ID: NP_207177.1), termed as FutA herein. FutA is encoded by the futA gene of H. pylori NCTC 26695.

In one embodiment, the heterologous α1,3/4-fucosyl transferase expressed comprises or preferably consists of a polypeptide that is identical with SEQ ID No. 4. The protein according to SEQ ID No. 4 is a functional variant of FutA in which Ala (A) at position 128 is substituted by Asn (N) and His (H) at position 129 is substituted by Glu (E) (Choi et al. Biotechnol. Bioengin. 113, 1666 (2016)). The protein according to SEQ ID No. 4 is termed as FutA_mut herein, and the nucleic acid sequence encoding FutA_mut is termed as futA_mut herein.

In one embodiment, the heterologous α1,3/4-fucosyl transferase expressed comprises or preferably consists of a polypeptide that is identical with SEQ ID No. 7. The protein according to SEQ ID No. 7 is a functional variant of FutA in which Ala (A) at position 128 is substituted by Asn (N), His (H) at position 129 is substituted by Glu (E), Asp (D) at position 148 is substituted by Gly (G) and Tyr (Y) at position 221 is substituted by Cys (C). The protein according to SEQ ID No. 7 is termed as FutA_mut2 herein, and the nucleic acid sequence encoding FutA_mut2 is termed as futA_mut2 herein.

In preferred embodiments, the heterologous α1,3/4-fucosyl transferase expressed comprises or preferably consists of a polypeptide that is identical with SEQ ID No. 1, SEQ ID No. 2, SEQ ID No. 3, SEQ ID No. 4 or SEQ ID No. 7.

The above genetically modified microorganism or cell, preferably, comprises a functional GDP-fucose metabolic pathway, and/or a lactose import system including an active transport mechanism mediated by a lactose permease, preferably that encoded by the lacY gene.

The term “microorganism” or “cell” in the present context designates a biological cell, e.g. a bacterial or yeast cell, that can be genetically manipulated to express its native or foreign genes, being as chromosome (chromosomal) gene or plasmid integrated (plasmid-borne) gene, at different expression levels.

The terms “host cell”, “recombinant microorganism or cell” or “genetically modified microorganism or cell” are used interchangeably to designate a cell, preferably a bacterial cell, that contains at least one artificial alteration in its genome compared to its naturally occurring (wild type) variant. By the alteration, either a nucleic acid construct is added to the cell by way of integration into the genome or by addition via plasmid, or a nucleic acid sequence is deleted from or changed in the genome of the cell. Whatever is the case, the so-transformed cell has a genotype that is different from that before the alteration and, therefore, the modified cell shows modified feature(s). Preferably, the genetically modified cell can perform at least one additional or altered biochemical reaction, when cultured or fermented, due to the introduction of a heterologous nucleic acid sequence or the modification of a native nucleic acid sequence that encodes an enzyme that is not expressed in the wild type cell, or the genetically modified cell cannot perform a biochemical reaction due to the deletion, addition or modification of a nucleic acid sequence that encodes an enzyme found in the wild type cell. The genetically modified cell can be constructed by well-known, conventional genetic engineering techniques (e.g. Green and Sambrook: Molecular Cloning: A laboratory Manual, 4^{th} ed., Cold Spring Harbor Laboratory Press (2012); Current protocols in molecular biology (Ausubel et al. eds.), John Wiley and Sons (2010)).

The term “sequence identity of [a certain] %” in the context of two or more nucleic acid or amino acid sequences means that the two or more sequences have nucleotides or amino acid residues in common in the given percent when compared and aligned for maximum correspondence over a comparison window or designated sequences of nucleic acids or amino acids (i.e. the sequences have at least 90 percent (%) identity). Percent identity of nucleic acid or amino acid sequences can be measured using a BLAST 2.0 sequence comparison algorithms with default parameters, or by manual alignment and visual inspection (see e.g. http://www.ncbi.nlm.nih.gov/BLAST/). This definition also applies to the complement of a test sequence and to sequences that have deletions and/or additions, as well as those that have substitutions. An example of an algorithm that is suitable for determining percent identity, sequence similarity and for alignment is the BLAST 2.2.2+ algorithm, which is described in Altschul et al. Nucl. Acids Res. 25, 3389 (1997). BLAST 2.2.20+ is used to determine percent sequence identity for the nucleic acids and proteins of the invention. Software for performing BLAST analyses is publicly available through the National Center for Biotechnology Information (http://www.ncbi.nlm.nih.gov/). Examples of sequence alignment algorithms are CLUSTAL Omega (http://www.ebi.ac.uk/Tools/msa/clustalo/), EMBOSS Needle (http://www.ebi.ac.uk/Tools/psa/emboss_needle/), MAFFT (http://mafft.cbrc.jp/alignment/server/) or MUSCLE (http://www.ebi.ac.uk/Tools/msa/muscle/).

In a preferred embodiment, the genetically modified cell of the invention has been transformed to contain a nucleic acid construct comprising a coding sequence for a protein, enzyme or polypeptide having a glycosyl transferase activity, preferably one or more constructs comprising one or more coding nucleic acid sequence(s) of one or more heterologous transferase(s), preferably at least one sequence encoding a β1,3-N-acetyl glucosaminyl transferase, at least one sequence encoding a β1,3-galactosyl transferase and at least one sequence encoding an α1,3/4-fucosyl transferase, and is capable of expressing the coding nucleic acid sequence comprised in the construct.

The genetically modified microorganism or cell of this invention can be selected from the group consisting of bacteria and yeasts, preferably a bacterium. Bacteria are preferably selected from the group of: Escherichia coli, Bacillus spp. (e.g. Bacillus subtil1is), Campylobacter pylori, Helicobacter pylori, Agrobacterium tumefaciens, Staphylococcus aureus, Thermophilus aquaticus, Azorhizobium caulinodans, Rhizobium leguminosarum, Neisseria gonorrhoeae, Neisseria meningitis, Lactobacillus spp., Lactococcus spp., Enterococcus spp., Bifidobacterium spp., Sporolactobacillus spp., Micromomospora spp., Micrococcus spp., Rhodococcus spp., Pseudomonas, among which E. coli is preferred.

The term “genetically modified microorganism or cell being capable of producing LNFP-V from lactose” means that said cell possesses enzymatic activity that is necessary for synthesizing LNFP-V, a pentasaccharide, from lactose, a disaccharide, via consecutive glycosylation steps, wherein lactose is glycosylated, in a first glycosylation step, to a trisaccharide, then that trisaccharide is glycosylated, in a second glycosylating step, to a tetrasaccharide, and at last that tetrasaccharide is glycosylated, in a third glycosylation step, to LNFP-V. The glycosylation steps are mediated by respective glycosyl transferases. In a glycosyl transferase mediated glycosylation, the glycosyl transferase in question transfers a monosaccharide of an appropriate donor molecule, the donor molecule being an activated monosaccharide nucleotide, to the acceptor molecule. The necessary glycosyl transferases according to the invention are: a β1,3-N-acetyl glucosaminyl transferase, a β1,3-galactosyl transferase and an α1,3/4-fucosyl transferase; and the corresponding donors are: UDP-GlcNAc, UDP-Gal and GDP-Fuc, respectively.

The production of UDP-GlcNAc and UDP-Gal by the cell takes place under the action of enzymes involved in their natural de novo biosynthetic pathways in stepwise reaction sequence starting from a simple carbon source like glycerol, fructose, sucrose or glucose (for a review for monosaccharide metabolism see e.g. H. H. Freeze and A. D. Elbein: Chapter 4: Glycosylation precursors, in: Essentials of Glycobiology, 2^{nd} edition (Eds. A. Varki et al.), Cold Spring Harbour Laboratory Press (2009)). Specifically, UDP-GlcNAc is produced de novo from fructose-6-phosphate in three steps catalyzed by the enzymes encoded by three genes, glmS, glmM and glmU, which are expressed under their native promoter. Similarly, UDP-Gal is produced de novo from glucose-6-phosphate in three steps catalyzed by the enzymes encoded by the genes pgm, galU and galE, which are also expressed under their native promoter. The GDP-Fuc metabolic pathway vide infra. According to a certain synthetic sequence to produce LNFP-V, lactose is N-acetylglucosaminylated to lacto-N-triose II (GlcNAcβ1-3Galβ1-4Glc), followed by galactosylation to LNT (Galβ1-3GlcNAcβ1-3Galβ1-4Glc), and at last by fucosylation to LNFP-V. However, it may be possible that the fucosylation precedes or follows the N-actylglucosaminylation step.

The term “a nucleic acid sequence [. . . ] being integrated in the genome of the microorganism or cell” means that said nucleic acid sequence, in its entirety, alone or comprised in a nucleic acid construct, preferably being operably linked to one or more control sequence(s) that is recognized by the host cell, is inserted in a certain site of the genome (genetic locus) of said microorganism or cell.

The term “nucleic acid sequence encoding a polypeptide having a β1,3-N-acetyl glucosaminyl transferase activity” or “nucleic acid sequence encoding a polypeptide having a β1,3-galactosyl transferase activity” means a gene, a functional fragment thereof or a codon-optimized version thereof that express a polypeptide having β1,3-N-acetyl glucosaminyl transferase activity or β1,3-galactosyl transferase activity, respectively.

The term “gene” in the present context relates to a coding nucleic acid sequence. A “functional fragment” or a “functional variant of a gene” preferably means a fragment of the coding sequence or a modified coding sequence, e.g. a sequence comprising one or more nucleotides that differ from the nucleotides at the same positions of the original coding sequence, that express a polypeptide having functional feature(s) that is identical or a similar to the polypeptide expressed from the original coding sequence.

The term “nucleic acid construct” means an artificially constructed segment of nucleic acids, in particular a DNA sequence, which is intended to be transplanted into a target cell, e.g. a bacterial cell. In the context of the invention, the nucleic acid construct contains a recombinant DNA sequence comprising a coding DNA sequence of the invention. In one preferred embodiment, the nucleic acid construct comprises essentially four isolated DNA sequences operably linked together: a coding DNA sequence, a promoter DNA sequence linked to the coding DNA sequence so that it is capable of initiating the transcription of said coding DNA sequence, a DNA fragment of a 5′-untranslated region (5′-UTR) located upstream of a gene, i.e. the gene leader DNA sequence directly upstream from the initiation codon and downstream the promoter sequence. The DNA construct of the invention may be inserted into a plasmid DNA/vector, transplanted into the target/host cell and expressed as plasmid- or chromosome-borne. The DNA construct may be linear or circular. A linear or circular DNA construct integrated into the host bacterial genome or expression plasmid is interchangeably termed herein as “expression cassette”, “expression cartridge” or “cartridge”. Preferably, the cartridge is a linear DNA construct comprising essentially sequences of a promoter, a 5′-UTR DNA (including a ribosomal binding site) downstream of the promoter, and operably linked to a coding DNA sequence encoding a biological molecule of interest. The construct may also comprise further sequences, such as a transcriptional terminator sequence, and two terminally flanking regions, which are homologous to a genomic region and which enable homologous recombination. In addition, the cartridge may contain other sequences as described below. The cartridge can be made by methods well-known known in the art, e.g. using standard methods described in Principles and techniques of biochemistry and molecular biology (Wilson and Walker, eds.), Cambridge University Press (2010). The use of a linear expression cartridge may provide the advantage that the genomic integration site can be freely chosen by the respective design of the flanking homologous regions of the cartridge. Thereby, integration of the linear expression cartridge allows for greater variability with regard to the genomic region. Since linear cartridges are also easier to construct, such cartridges are preferred embodiments of the construct of the invention.

The term “promoter” means a nucleic acid sequence involved in the binding of RNA polymerase to initiate transcription of an operably linked gene, wherein the gene includes a coding DNA sequence and other (non-coding) sequences, e.g. the 5′-untranslated region (5′-UTR) located upstream of the coding sequence, which comprises a ribosomal binding site. A promoter in this invention is an isolated DNA sequence, i.e. not an integrated DNA fragment of the genomic DNA. The nucleotide sequence of a promoter of the invention corresponds to, or have at least 80% identity, preferably 90-99.9% identity with the nucleotide sequence of a fragment of bacterial genomic DNA that is regarded as promoter region of a gene, e.g. a promoter region of a glp operon or lac operon of E.coli. By “operon” is meant a functioning unit of genomic DNA containing a cluster of genes under the control of a single promoter. By “glp operon” is meant a cluster of genes involved in the respiratory metabolism of glycerol of bacteria. By “lac operon” is meant a cluster of genes involved in transport and metabolism of lactose. The invention in preferred embodiments refers to four glp operons of E. coli, in particular, glpFKX, glpABC, glpTQ, and glpD. In other preferred embodiments, the invention refers to lac operon of E. coli comprising genes Z, Y and A. Preferably, a glp operon promoter sequence comprised in a DNA construct of the invention corresponds to or has at least 80% identity, preferably 90-99.9% identity with the nucleotide sequence of a fragment of the genomic DNA regarded as a promoter region of the corresponding glp operon of E. coli; in particular, the isolated sequence of a promoter of a glp operon of the invention corresponds to, or has said percent of identity with a fragment of the genomic sequence upstream the sequence having GenBank ID: EG10396 (glpFKX), EG10391 (glpABC), EG10394 (glpD), EG10401 (glpTQ); and an isolated sequence of promoter of operon lacZYA corresponds to, or has said percent of identity with, a fragment of the genomic sequence upstream the sequence having GenBank ID: EG10527 (lacZ). The E. coli genome is referred herein to the complete genomic DNA sequence of E coli K-12 MG1655 (GenBank ID:U00096.3).

A promoter sequence in this invention may comprise several structural features/elements, such as regulatory regions capable of binding RNA polymerase in a cell and initiating transcription of a downstream (3′-direction) coding sequence, the transcriptional start site and binding sites for their specific transcriptional regulator protein. The regulatory region comprises protein binding domains (consensus sequences) responsible for the binding of RNA polymerase such as the -35 box and the -10 box (Pribnow box).

A promoter sequence in this invention preferably comprises at least 90 nucleotides, more preferably from 100 to 150 nucleotides, e.g. 110-120, 120-130, 130-140, 140-150, or even more preferably over 150 nucleotides, such as 155-165, 165-175, 175-185, 185-195, 195-205, 205-215, 215-225, 225-235, 235-245, 245-255, 255-265. In some embodiments, the promoter sequence may be even longer, such as up to 500-1000 nucleotide long. In some preferred embodiments, an isolated sequence of the promoter is that of the glp operon.

The invention also relates to variants of the promoter DNA sequences included in the construct of the invention. By “variant” in the present content is meant an artificial nucleic acid sequence that has 70-99.9% similarity to a nucleotide sequence of the concerned promoter DNA sequence. The percentage of similarity of compared nucleic acid sequences indicates the portion of the sequences that has identical structure i.e. identical nucleotide composition. The percentage of sequence similarity for the purposes of the invention can be determined by using any method well-known in the art e.g. BLAST. The scope of the term “variant” includes nucleotide sequences complementary to the DNA sequences described herein, mRNA sequences and synthetic nucleotide sequences, e.g. PCR primers, and other oligonucleotides which relate to the nucleic acid sequences of constructs of the invention.

In a preferred embodiment, a promoter of the glp operon or its variant as disclosed above is operably linked to the heterologous β1,3-N-acetyl glucosaminyl transferase, the heterologous β1,3-galactosyl transferase and/or the heterologous α1,3/4-fucosyl transferase. More preferably, the promoter of the glp operon is the glpF promoter or its variant.

Also, preferably, the heterologous β1,3-N-acetyl glucosaminyl transferase, the heterologous β1,3-galactosyl transferase and the heterologous α1,3/4-fucosyl transferase, which are necessary for the synthesis of LNFP-V, are expressed under a glpF promoter variant. In this regard, expression cassettes are constructed, each comprising the heterologous β1,3-N-acetyl glucosaminyl transferase, the heterologous β1,3-galactosyl transferase or the heterologous α1,3/4-fucosyl transferase, respectively, operably linked to the glpF promoter variant, and inserted into the genome of the host cell. The glpF promoter variant, preferably, is a nucleic acid construct characterized by SEQ ID No. 5.

The term “microorganism or cell comprises a functional GDP-fucose metabolic pathway” means that said cell or microorganism is able to produce GDP-fucose in situ that serves as fucose donor in the fucosylation step to make LNFP-V within the microorganism or cell. The GDP- fucose metabolic pathway is either a de novo synthesis or occurs according to a salvage pathway. In the de novo pathway, GDP-L-fucose is biosynthesized from fructose-6-phosphate and GTP by the successive action of five enzymes: mannose-6-phosphate isomerase, phosphomannomutase, mannose-1-phosphate guanylyl transferase, GDP-mannose-4,6-dehydratase and GDP-fucose synthase. In E. coli, these five enzymes are encoded by manA, manB, manC, gmd and wcaG, respectively, genes which are part of the colanic acid gene cluster. Other bacterial or yeast strains may lack one or more of the enzymes mentioned above; any enzymes in the de novo GDP-fucose synthesis pathway that are inherently missing can be provided as genes or recombinant DNA constructs, either in a plasmid expression vector or as exogenous genes integrated in the chromosome of the host cell. In addition, the wcaJ gene of the colanic acid cluster encoding UDP-glucose lipid carrier transferase shall be deleted or inactivated in order to suppress the production of colanic acid and thus the GDP-fucose biosynthesis flux to be diverted from it to the synthesis of LNFP-V. Preferably, the homologous colanic acid cluster disclosed above is under the control of the lac promoter (Plac). In addition, further to enhance the GDP-fucose pool, the gene rscA that encodes a positive regulator of the colanic acid operon may be overexpressed (see e.g. Dumon et al. Glycoconj. J. 18, 465 (2001)). In another embodiment, the genetically modified cell can utilize salvaged fucose for producing GDP-fucose. In the salvage pathway, exogenously added fucose, internalized in the cell with the aid of fucose permease, is phosphorylated by fucose kinase and converted to GDP-fucose by fucose-1-phosphate guanylyl transferase. The enzymes involved in the procedure can be heterologous or homologous ones. In one embodiment, the fucose kinase and the fucose-1-phosphate guanylyl transferase can be combined in a bifunctional enzyme (see e.g. WO 2010/070104).

The term “lactose import system comprising an active transport mechanism mediated by a lactose permease” means that lactose necessary for making LNFP-V and added exogenously to the culture is internalized with the aid of an active transport comprising a transporter protein having specificity towards lactose, called lactose permease, thereby the genetically modified cell or microorganism admits and concentrates the exogenous lactose in its cytoplasm. The internalization cannot affect the basic and vital functions or destroy the integrity of the cell. A generally recognized and widely used permease for importing lactose into the cell is LacY (see e.g. WO 01/04341), though other permeases having specificity towards lactose may also be considered.

The genetically modified microorganism or cell able to produce LNFP-V from lactose disclosed herein comprises, in a preferred embodiment, a heterologous nucleic acid sequence encoding a polypeptide having an α1,3/4-fucosyl transferase activity selected from the group consisting of protein of SEQ ID No. 1 (Ma et al. J. Biol. Chem. 281, 6385 (2006)), protein of SEQ ID No. 2 (Gen Bank ID: AAB81031.1, Ge et al. J. Biol. Chem. 272, 21357 (1997)), protein of SEQ ID No. 3 (Gen Bank ID: NP_207177.1), protein of SEQ ID No. 4 (Choi at al. Biotechnol. Bioeng. 113, 1666 (2016)) or protein of SEQ ID No. 7.

Preferably, the heterologous nucleic acid sequences encoding a polypeptide comprising or consisting of an amino acid sequence that has a sequence identity of at least 90% with amino acid sequence of SEQ ID No. 1 or SEQ ID No. 3, advantageously those encoding the protein of SEQ ID No. 1 (called “truncated” fucT), the protein of SEQ ID No. 2 (called fucT), the protein of SEQ ID No. 3 (called futA), the protein of SEQ ID No. 4 (called futA_mut) or the protein of SEQ ID No. 7 (called futA_mut2) are codon-optimized for the expression system of the invention.

Preferably, the genetically modified microorganism or cell disclosed above is not able to hydrolyse or degrade LNFP-V or its intermediates in the biosynthetic pathway starting from lactose (like lacto-N-triose II or LNT). Likewise, in one embodiment, the cell lacks any enzyme activity, such as LacZ (β-galactosidase) activity, that would degrade the acceptor (lactose). This can be achieved by deletion or inactivation of lacZencoding β-galactosidase. In other embodiment, the genetically modified microorganism or cell disclosed above, although its native lacZ is deleted or deactivated, still may have a low level of β-galactosidase activity, e.g. due to incorporation of a heterologous gene encoding a β-galactosidase. In this regard, the excess of lactose that is added exogenously may be completely hydrolysed after fermentation, thereby facilitating the isolation and purification of the produced oligosaccharides of interest. Such a solution is disclosed e.g. in WO 2012/112777. In other embodiment, the genetically modified microorganism or cell disclosed above may be additionally altered to comprise a 13- galactosidase gene which is operably linked to an inducible promoter, e.g. a temperature inducible promoter. In this regard, the β-galactosidase is not expressed in lower temperature, e.g. at the temperature at which the microorganism is cultured to produce LNFP-V, while it's expression is induced in the end of fermentation upon raising the temperature and, thus, the excess of lactose can be hydrolysed. Such a solution is disclosed e.g. in WO 2015/036138.

Also, preferably, the nucleic acid sequence encoding a polypeptide having a β1,3-N-acetyl glucosaminyl transferase activity that is integrated in the genome of the genetically modified microorganism or cell is the lgtA gene of Neisseria meningitidis 053442 (GenBank ID: CP000381) or a codon-optimized version thereof. Preferably, the coding sequence of the lgtA gene or a codon-optimized version thereof is operably linked to, thereby expressed under, the glpF promoter variant characterized by SEQ ID No. 5.

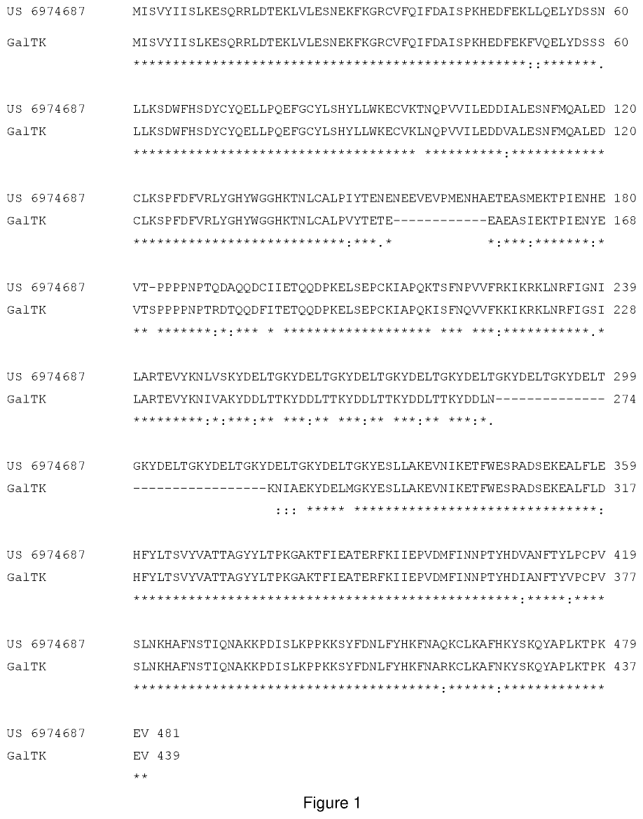

Also, preferably, the nucleic acid sequence encoding a polypeptide having a β1,3-galactosyl transferase activity that is integrated in the genome of the genetically modified microorganism or cell is a gene termed as galTK, a functional fragment thereof or a codon-optimized version thereof. galTK is homologous to a gene of H. pylori 43504 encoding a β1,3-galactosyl transferase (GenBank ID: BD182026, U.S. Pat. No. 6,974,687) and encodes the protein characterized by SEQ ID No. 6, which is termed as GalTK. The structural comparison of the β1,3-galactosyl transferase disclosed by U.S. Pat. No. 6,974,687 with the GalTK β1,3-galactosyl transferase used in the present application (encoded by galTK) is shown in FIG. 1.

According to the invention the genetically modified microorganism or cell described herein, including the preferred and more preferred embodiments, provides a sufficient amount of GDP-fucose for the biosynthesis of LNFP-V either by the de novo or the salvage pathway, preferably by the de novo pathway (vide supra). However, to further optimize the LNFP-V biosynthesis by providing the necessary and sufficient GDP-fucose level, an additional copy of the colanic acid gene cluster may be introduced into the cell, preferably incorporated in the genome of the cell.

The genetically modified microorganism or cell disclosed herein, including the preferred and more preferred embodiments, comprises a heterologous β1,3-N-acetyl glucosaminyl transferase, a heterologous β1,3-galactosyl transferase and a heterologous α1,3/4-fucosyl transferase integrated into the genome of the cell. The later heterologous genes may be integrated in any genetic locus of the host cell so that cellular metabolism in not disturbed and the cell is capable of producing the desired oligosaccharide. The expression system used or suitable in the invention allows a wide variability. In principle, any locus with known sequence may be chosen, with the proviso that the function of the sequence is either dispensable or, if essential, can be complemented (as e.g. in the case of an auxotrophy). Many integration loci suitable for the purposes of the invention are described in the prior art (see e.g. Francia et al. J. Bacteriol. 178, 894 (1996): Juhas et al. (2014) PLoS ONE 9, e111451 (2014); Juhas et al. (2015) Microbial Biothechnol. 8, 617 (2015); Sabi et al. Microbial. Cell Factories 12:60 (2013)).

Preferably, the genomic sites of integration are, in one embodiment, loci in an operon of sugar metabolic genes, such as those of galactose, xylose, ribose, maltose or fucose.

According to the invention, the genetically modified microorganism or cell, including any of the preferred embodiments disclosed above, in one embodiment, comprises only one (single) copy of a β1,3-galactosyl transferase gene, preferably galTK or a codon-optimized version thereof, more preferably under the control of a glp promoter (Pglp) or a variant thereof, e.g. PglpF, especially the PglpF variant according to SEQ ID No. 5.

According to the invention, the genetically modified microorganism or cell, including any of the preferred embodiments disclosed above, in one embodiment, comprises only one (single) copy of a β1,3-N-acetyl glucosaminyl transferase gene, preferably the coding sequence of the lgtA gene or a codon-optimized version thereof, more preferably under the control of a glp promoter (Pglp) or a variant thereof, e.g. PglpF, especially the PglpF variant according to SEQ ID No. 5.

The genetically modified microorganism or cell, including any of the preferred embodiments disclosed above, in one embodiment, comprises only one (single) copy of each of the β1,3-galactosyl transferase gene, preferably galTK or a codon-optimized version thereof, the β1,3-N-acetyl glucosaminyl transferase gene, preferably the coding sequence of the lgtA gene or a codon-optimized version thereof, and the α1,3/4-fucosyl transferase gene encoding a polypeptide having an amino acid sequence identity of at least 90% with SEQ ID No. 1 or SEQ ID No. 3, preferably “truncated” fucT, fucT, futA, futA_mutor futA_mut2. More preferably, each such glycosyl transferase is under the control of the glpF promoter variant according to SEQ ID No. 5. Each copy of the different glycosyl transferases genes is integrated in different genomic sites, preferably in loci associated with utilization of alternative carbon sources.

In one embodiment, the genetically modified microorganism or cell, including any of the preferred embodiments disclosed above, comprises two copies of the (31,3-galactosyl transferase gene, preferably galTK or a codon-optimized version thereof, each of which is integrated in two different genomic sites, and two copies of the α1,3/4-fucosyl transferase gene encoding a polypeptide having an amino acid sequence identity of at least 90% with SEQ ID No. 1 or SEQ ID No. 3, preferably “truncated” fucT, fucT, futA, futA_mutor futA_mut2, each of which is integrated in two different genomic sites. More preferably, each such glycosyl transferase coding sequence is expressed under the control of PglpF or another glp promoter or a variant thereof, e.g. PglpA or PglpT, preferably under the control of the glpF promoter variant according to SEQ ID No. 5.

The three different kinds of heterologous glycosyl transferase genes described above that are necessary for the production of LNFP-V are incorporated in the genome of the host cell as a part of an expression cassette, that is in the form of a DNA construct that contains the glycosyl transferase coding sequence operably linked to a promoter sequence. The promoter may be any suitable promoter that is capable of initiating and maintaining the transcription of the operably linked gene on a certain level and recognized by the host cell. Preferably, the promoter is a carbon source inducible promoter. Preferably, a promoter is one naturally regulating the transcription of genes of one of four glp operons, glpFKX, glpABC, glpTC? and glpD, of E. coli, or variants thereof.

The genetically modified microorganism or cell, including the preferred and more preferred embodiments disclosed above, in one embodiment, comprise only one (single) copy of an α1,3/4-fucosyl transferase selected from the group consisting of fucT, futA, futA_mut and futA_mut2, preferably under the control of a glp promoter, more preferably under the control of the glpF promoter or a variant thereof, even more preferably under the control of the glpF promoter variant according to SEQ ID No. 5.

As disclosed above, the genetically modified microorganism or cell suitable for making LNFP-V from lactose according to the invention, in one embodiment, may comprise a GDP-fucose de novo biosynthetic pathway to provide GDP-fucose intracellularly. The de novo pathway to GDP-fucose utilizes the native colanic acid gene cluster of the host cell, preferably E. coli, comprising manA, manB, manC, gmd and wcaG, and wherein wcaJ is deleted or deactivated. The native colanic acid gene cluster is preferably under the control of the Plac promoter. In a further embodiment, the genetically modified microorganism or cell of the invention, besides the native colanic acid gene cluster, may comprise an additional copy of colanic acid genes to enhance the GDP-fucose biosynthesis and thereby providing a higher level of GDP-fucose. Such a second copy of the colanic acid gene cluster is preferably integrated in the genome, and preferably expressed under the control of a glp promoter, more preferably under the glpF promoter or a variant thereof,even more preferably under the control of the glpF promoter variant according to SEQ ID No. 5. As to the genomic site in which the second copy of the colanic acid gene cluster is incorporated, it can preferably be the loci of the cell's sugar metabolic genes as disclosed above. In another embodiment, when the cell comprises an additional copy of the colanic acid gene cluster, only one (single) genomic copy of the α1,3/4-fucosyl transferase gene, preferably futA_mut or futA_mut2, more preferably futA_mut2, is present.

A second aspect of the invention relates to a method for producing LNFP-V, the method comprising:
a) providing a genetically modified microorganism or cell as disclosed in the first aspect of the invention,
b) culturing said microorganism or cell in the presence of lactose, and
c) separating LNFP-V from said microorganism or cell, from the culture medium or from both.

The pentasaccharide LNFP-V can be readily obtained by a process which involves culturing or fermenting a genetically modified cell or microorganism according to the first aspect of the invention in an aqueous culture medium or fermentation medium containing lactose and one or more carbon-based substrates followed by separating them from the culture medium. By the term “culture medium” is meant the aqueous environment of the fermentation process in a fermenter outside of the genetically modified cell.

In carrying out this process, the genetically modified cell is cultured in the presence of a carbon- based substrate such as glycerol, glucose, sucrose, glycogen, fructose, maltose, starch, cellulose, pectin, chitin, etc. Preferably, the cell is cultured with glycerol, glucose, sucrose and/or fructose.

This process also involves initially transporting the exogenous lactose from the culture medium into the genetically modified cell. Lactose is added exogenously in a conventional manner to the culture medium, from which it is transported into the cell. The lactose is internalized with the aid of an active transport mechanism, by which lactose diffuses across the plasma membrane of the cell under the influence of a transporter protein or lactose permease (LacY) of the cell, which is expressed under the control of the lac promoter.

In some embodiments, the genetically modified cell used in this process lacks enzymatic activity which would significantly degrade intracellular lactose, LNFP-V and the metabolic intermediates in the LNFP-V biosynthetic pathway, for example lacto-N-triose II or LNT. In this regard, the native β-galactosidase of the cultured cell (encoded by the lacZgene in E. coli), which hydrolyses lactose to galactose and glucose, is preferably deleted or inactivated (LacZ⁻ genotype). In one embodiment of the second aspect of the invention, excess of lactose added in step b) is not removed or degraded after fermentation and a mixture of lactose and LNFP-V, optionally accompanied by one or more oligosaccharide by-products such as e.g. lacto-N-triose II, LNT, 3-FL and/or LNDFH-II, is separated and isolated from the culture medium. In another embodiment, a mixture of lactose and LNFP-V, optionally accompanied by one or more oligosaccharide by-products such as e.g. lacto-N-triose II, LNT, 3-FL and/or LNDFH-II, is produced by fermentation as above, and LNFP-V, optionally accompanied by one or more oligosaccharide by-products such as e.g. lacto-N-triose II, LNT, 3-FL and/or LNDFH-II, is separated and isolated from the culture milieu and optionally from the excess of lactose. Yet in other embodiment, a mixture of lactose and LNFP-V, optionally accompanied by one or more oligosaccharide by-products such as e.g. lacto-N-triose II, LNT, 3-FL and/or LNDFH-II, is produced by fermentation as above, and followed by
i) addition of a lactose degrading enzyme, e.g. a galactosidase, exogenously which hydrolyses lactose into monosaccharides, or
ii) letting the fermentation continue until all lactose added in the fermentation step is consumed,
   providing a substantially lactose-free broth. In this regard, in option ii), a genetically modified cell of LacZ⁻ genotype disclosed above can further comprise a functional recombinant β-galactosidase. This functional galactosidase may be encoded by an exogenous /acZgene which is heat inducible (see e.g. WO 2015/036138). At the temperature of the fermentation this functional β-galactosidase is not expressed by the cell, therefore the internalized lactose is not degraded in the cell while the HMOs are produced. When the desired concentration or amount of LNFP-V in the broth is reached, the culturing is continued at elevated temperature by which the functional β-galactosidase is expressed and the lactose in excess is degraded. Alternatively, a genetically modified cell of LacZ⁻ genotype disclosed above may comprise a recombinant β-galactosidase of low but detectable level of activity (see e.g. WO 2012/112777) in order to remove the optional residual lactose at the end of fermentation.

Typically, the process involves providing, in the culture medium, a carbon-based substrate and at least 30, up to about 100, grams of lactose per litre of the initial volume of the culture medium. Preferably, the process is also carried out at a temperature of 28 to 35° C., preferably with continuous agitation and continuous aeration for 2 to 5 days. It is preferred that the final volume of the culture medium is not more than three-fold of the volume of the initial volume of the culture medium before providing lactose and the carbon-based substrate to the culture medium.

According to an embodiment in carrying out the process of the invention, a genetically modified LacZ⁻Y⁺ E. coli strain is cultured in the following way:
(1) a first phase of exponential cell growth that is ensured by a carbon-based substrate, such as glucose or sucrose, provided in the culture medium and that preferably lasts until the glucose has all been consumed which is preferably at least 12 hours, such as around 18 hours or 20-25 hours; and
(2) a second phase of cell growth that is limited by a carbon-based substrate, such as glucose, sucrose or glycerol, and lactose which are provided, preferably continuously, in the culture medium after the first phase and that lasts until the carbon-based substrate and preferably most (e.g. at least 60%) of the lactose have been consumed which is preferably at least 35 hours, such as at least 45 hours, 50 to 70 hours, or up to about 130 hours.

During culturing of the genetically modified cell, LNFP-V and optionally one or more oligosaccharide by-products such as e.g. lacto-N-triose II, LNT, 3-FL and/or LNDFH-II, accumulate in both the cell's intracellular and extracellular matrices. The oligosaccharides produced can be isolated from the broth and/or separated from each other by using standard techniques.

A third aspect of the invention relates to a protein (polypeptide) that comprises or consists of the amino acid sequence characterized by SEQ ID No. 7. The protein (polypeptide) that comprises or consists of the amino acid sequence characterized by SEQ ID No. 7 has α1,3/4-fucosyl transferase activity and can be used advantageously in the fermentative production of LNFP-V as disclosed in the examples.

A fourth aspect of the invention relates to use of a polypeptide having an α1,3/4-fucosyl transferase activity in the production of LNFP-V, the polypeptide is selected from the group consisting of:
a polypeptide comprising or consisting of an amino acid sequence that has a sequence identity of at least 90% with amino acid sequence of SEQ ID No. 1, and
a polypeptide comprising or consisting of an amino acid sequence that has a sequence identity of at least 90% with amino acid sequence of SEQ ID No. 3.

In certain embodiments, the α1,3/4-fucosyl transferase comprises or consists of a polypeptide that is identical with SEQ ID No. 1 or SEQ ID No. 3, whichever the case may be, in at least 92%, in at least 94%, in at least 95%, in at least 96%, in at least 97%, in at least 98% or in at least 99%.

In one embodiment, the α1,3/4-fucosyl transferase comprises, preferably consists of, the amino acid sequence of SEQ ID No 1 or SEQ ID No. 2.

In one embodiment, the α1,3/4-fucosyl transferase comprises, preferably consists of, the amino acid sequence of SEQ ID No 3, SEQ ID No. 4 or SEQ ID No. 7.

In a preferred embodiment, the α1,3/4-fucosyl transferase comprises or preferably consists of a polypeptide that is identical with SEQ ID No. 7.

In one embodiment, a nucleic acid sequence that encodes the polypeptide having an α1,3/4-fucosyl transferase activity is comprised in a microorganism or cell that is able to produce LNFP-V from lactose. The nucleic acid sequence can be introduced into the microorganism or cell by using an appropriate expression plasmid or via genome (chromosome) integration.

### EXAMPLES

In the examples, all utilized strains are derived from an E. coli platform strain that was constructed from E. coli K12 DH1 (genotype: F⁻, λ⁻, gyrA96, recA1, relA1, endA1, thi-1, hsdR17, supE44, obtained from Deutsche Sammlung von Mikroorganismen and Zellkulturen (DSMZ), www.dsmz.de, reference DSM 4235) by disrupting (deletions of) the genes lacZ, nanKETA, lacA, melA, wcaJ, mdoH and by inserting a Plac promoter upstream the gmd gene.

Gene targeting in the chromosomal DNA was done using standard DNA manipulation techniques, e.g. as disclosed in Warming et al. Nucleic Acids Res. 33, e36 (2005). Insertion of genetic cassettes in the chromosomal DNA was done by gene Gorging as described by Herring et al. Gene 311, 153 (2003).

The strains disclosed in the examples were screened in 24 deep well plates using a 4-day protocol. During the first 24 hours, cells were grown to high densities while in the next 72 hours cells were transferred to a medium that allowed induction of gene expression and product formation. Specifically, during day 1 fresh inoculums were prepared using a basal minimal medium supplemented with magnesium sulphate, thiamine and glucose. After 24 hours of incubation of the prepared cultures at 34° C. with a 700 rpm shaking, cells were transferred to a new basal minimal medium (2 ml) supplemented with magnesium sulphate and thiamine to which an initial bolus of 20% glucose solution (1 μl) and 10% lactose solution (0.1 ml) were added, then 50% sucrose solution as carbon source was provided to the cells accompanied by the addition of sucrose hydrolase (invertase, 4 μl of a 0.1 g/I solution) so that glucose was provided at a slow rate for growth by cleavage of sucrose by the invertase. After inoculation of the new medium, cells were shaken at 700 rpm at 28° C. for 72 hours. After denaturation and subsequent centrifugation, the supernatants were analysed by HPLC.

### Example 1

E. coli platform strain (see above) was further modified as follows: a single copy of codon optimized lgtA coding sequence for LgtA was integrated into the genome (chromosome) of the E. coli platform strain in a locus related to sugar metabolism and expressed under the control of the glpF promoter; a single copy of codon optimized galTKwas integrated into the genome of the E. coli platform strain in another locus involved in sugar metabolism and expressed under the control of the glpF promoter; an additional copy of the colanic acid cluster was integrated in a third locus involved in the utilization of alternative carbon sources and expressed under the control of the glpF promoter; and lacI was deleted from the lac operon (ΔlacI). Based on the above strain, strains 1-5 were constructed by integrating a single copy of a gene encoding an α1,3/4-fucosyl transferase under the control of the glpF promoter in one of the loci of the E. coli platform strain that enables sugar metabolism:
strain 1: codon optimized futA sequence encoding the protein of SEQ ID No. 3
strain 2: codon optimized futA_mut2 sequence encoding the protein of SEQ ID No. 7
strain 3: codon optimized truncated fucT sequence encoding the protein of SEQ ID No. 1
strain 4: codon optimized fucT/// from H. pylori DSM 6709, GenBank ID: AY450598.1 (Rabbani et al. Glycobiology 15, 1076 (2005))
strain 5: codon optimized fucTa from H. pylori UA948, GenBank ID: AF194963.2.

After culturing, the following concentrations of LNFP-V were measured (intra- and extracellular concentrations together):

| | strain 1 | strain 2 | strain 3 | strain 4 | strain 5 |
|---|---|---|---|---|---|
| | futA | futA_mut2 | truncated fucT | fucTIII | fucTa |
| LNFP-V [nM] | 1.86 | 1.18 | 1.46 | 0.47 | 0 |

As shown in the table above, strains 1-3 expressing FutA, FutA_mut2 and truncated FucT α1,3/4-fucosyl transferase, respectively, produced much higher LNFP-V titers than reference strain 4 expressing FucTIII enzyme known from prior art in such constructs (by 300%, 150% and 210%, respectively). Reference strain 5 expressing FucTa α1,3/4-fucosyl transferase did not produce LNFP-V.

### Example 2

Based on strain 1 disclosed in Example 1, strain 6 was created so that it contained an additional (second) copy of codon optimized futA gene integrated in a sugar utilization locus and expressed under the control of the glpF promoter.

Similarly, based on strain 3 disclosed in Example 1, strain 7 was created so that it contained an additional (second) copy of codon optimized truncated fucT gene integrated in another sugar utilization locus and expressed under the control of the glpF promoter.

E. coli platform strain (see above) was further modified to make strain 8 as follows: a single genomic copy of codon optimized lgtA coding sequence was integrated in a locus involved in sugar consumption and expressed under the control of the glpF promoter; a single genomic copy of codon optimized galTK was integrated in another sugar metabolism locus and expressed under the control of the glpF promoter; a single genomic copy of codon optimized futA_mut2 was integrated in a locus enabling the utilization of another alternative carbon source and expressed under the control of the glpF promoter; an additional copy of the colanic acid cluster was integrated in a fourth sugar metabolism locus and expressed under the control of the glpF promoter; and lacI was deleted from the lac operon (ΔlacI). Based on strain 8, strain 9 was created so that it contained an additional (second) copy of codon optimized futA_mut2 gene integrated in a locus involved in a sugar consumption and expressed under the control of the glpF promoter.

After culturing, the following relative concentrations of LNFP-V were measured (intra- and extracellular concentrations together):

| | | | | | strain 3 | strain 7 |
|---|---|---|---|---|---|---|
| | strain 1 | strain 6 | strain 8 (1x | strain 9 (2x | (1x truncated | (2x truncated |
| | (1x futA) | (2x futA) | futA_mut2) | futA_mut2) | fucT) | fucT) |
| rel. LNFP-V | 100% | 104% | 100% | 65% | 100% | 113% |
| conc. | | | | | | |

As shown in the table above, the incorporation of a second copy of futA or truncated fucT encoding an α1,3/4-fucosyl transferase did not enhance the LNFP-V titer significantly, whereas the second copy of futA_mut2 had a negative impact on the LNFP-V titer. In conclusion, strains bearing a single genomic copy an α1,3/4-fucosyl transferase gene are preferable.

### Example 3

E. coli platform strain (see above) was further modified to make strain 10 as follows: a single genomic copy of codon optimized lgtA coding sequence was integrated in a sugar metabolism locus and expressed under the control of the glpF promoter; a single genomic copy of codon optimized galTK was integrated in another locus involved in alternative carbon source utilization and expressed under the control of the glpF promoter; a single genomic copy of codon optimized futA_mut2 was integrated in a third locus related to sugar consumption and expressed under the control of the glpF promoter; and lacl was deleted by replacement of galK (lacI::galK).

Based on strain 10, strains 11-13 were constructed as follows:
strain 11: an additional copy of the colanic acid cluster was integrated in a sugar utilization locus and expressed under the control of the glpF promoter;
strain 12: an additional (second) copy of codon optimized futA_mut2 gene was integrated in an another sugar metabolism locus and expressed under the control of the glpF promoter;
strain 13: an additional copy of the colanic acid cluster was integrated in a locus involved in utilization of alternative carbon source and expressed under the control of the glpF promoter, and additional (second) copy of codon optimized futA_mut2 gene was integrated in a locus that also enables the consumption of a specific sugar and expressed under the control of the glpF promoter.

After culturing, the following relative concentrations of LNFP-V were measured (intra- and extracellular concentrations together):

| | strain 10 | strain 11 | strain 12 | strain 13 |
|---|---|---|---|---|
| | 1x futA_mut2 | 1x futA_mut2 + CA | 2x futA_mut2 | 2x futA_mut2 + CA |
| rel. LNFP-V cone. | 100% | 117% | 94% | 64% |

Cell expressing an additional copy of the CA gene cluster and α1,3/4-fucosyl transferase from a single copy (strain 11) gave higher LNFP-V concentration (by ˜17%) than similar cell that did not have this additional PglpF-driven CA gene copy (strain 10). Markedly, the addition of a second genomic copy of the futA_mut2 gene does not improve the observed LNFP-V titers, regardless of the CA gene copy number.

### Example 4

Based on strain 8 disclosed in Example 2, strain 14 was created so that it contained an additional (second) copy of codon optimized galTK gene integrated in a locus involved in the metabolism of a given carbon source and expressed under the control of the glpF promoter.

Similarly, based on strain 9 disclosed in Example 2, strain 15 was created so that it contained an additional (second) copy of codon optimized galTK gene integrated in a locus in the E. coli platform strain and expressed under the control of the glpF promoter.

After culturing, the following relative concentrations of LNFP-V were measured (intra- and extracellular concentrations together):

| | strain 8 | strain 14 |
|---|---|---|
| | 1x futA_mut2 + 1x galTK | 1x futA_mut2 + 2x galTK |
| rel. LNFP-V conc. | 100% | 99% |
| | strain 9 | strain 15 |
| | 2x futA_mut2 + 1x galTK | 2x futA_mut2 + 2x galTK |
| rel. LNFP-V conc. | 100% | 174% |

The addition of a second β1,3-galactosyl transferase gene copy to strain 8 having a single copy of α1,3/4-fucosyl transferase gene had no effect on the final LNFP-V. However, the LNFP-V titer increased markedly when a second β1,3-galactosyl transferase gene copy was added to strain 9 which comprised 2 copies of the α1,3/4-fucosyl transferase gene.

1. A recombinant microorganism capable of producing LNFP-V from lactose, comprising:
a genome integrated heterologous nucleic acid sequence encoding a polypeptide having a β1,3-N-acetyl glucosaminyl transferase activity,
a genome integrated heterologous nucleic acid sequence encoding a polypeptide having a β1,3-_{g}alactosyl transferase activity, and
a genome integrated heterologous nucleic acid sequence encoding a polypeptide having an α1,3/4-fucosyl transferase activity,
wherein the polypeptide having an α1,3/4-fucosyl transferase activity is selected from the group consisting of:
a polypeptide comprising or consisting of an amino acid sequence that has a sequence identity of at least 90% with amino acid sequence of SEQ ID No. 1, and
a polypeptide comprising or consisting of an amino acid sequence that has a sequence identity of at least 90% with amino acid sequence of SEQ ID No. 3.

2. The microorganism according to claim 1, wherein the polypeptide having an α1,3/4-fucosyl transferase activity is selected from the group consisting of:
a protein that comprises the amino acid sequence set forth in SEQ ID No. 1,
a protein that comprises the amino acid sequence set forth in SEQ ID No. 3,
a protein that comprises the amino acid sequence set forth in SEQ ID No. 4, and
a protein that comprises the amino acid sequence set forth in SEQ ID No. 7.

3. The microorganism according to claim 1, wherein the polypeptide having an α1,3/4-fucosyl transferase activity is a protein that comprises the amino acid sequence set forth in SEQ ID No. 7.

4. The microorganism according to claim 1, further comprising a functional GDP-fucose biosynthetic pathway.

5. The microorganism according to claim 1, wherein the β1,3-N-acetylglucosaminyl transferase is encoded by the lgtA gene of Neisseria meningitidis 053442 or a codon-optimized version thereof, and/or the β1,3-galactosyl transferase is a protein that comprises the amino acid sequence set forth incharacterized by SEQ ID No. 6.

6. The microorganism according to claim 1, wherein the heterologous nucleic acid sequences are expressed under control of a glp promoter variant according to SEQ ID No. 5.

7. The microorganism according to claim 6, wherein the glp promoter is glpF or its variant according to SEQ ID No. 5.

8. The microorganism according to claim 1, wherein each heterologous nucleic acid sequence is integrated in the genome of the microorganism in a single copy.

9. The microorganism according to claim 1, wherein the polypeptide having an α1,3/4-fucosyl transferase activity is a protein that comprises the amino acid sequence set forth in SEQ ID No. 7 and its coding nucleic acid sequence is integrated in the genome of the microorganism in at least two copies, and wherein the β1,3-galactosyl transferase is a protein that comprises the amino acid sequence set forth in SEQ ID No. 6 and its coding nucleic acid sequence is integrated in the genome of the microorganism in at least two copies.

10. The microorganism according to claim 1, wherein at least one heterologous nucleic acid sequence is integrated in a site of an operon related to a sugar metabolism.

11. The microorganism according to claim 4, wherein the GDP-fucose biosynthetic pathway comprises recombinant manA, manB, manC, gmd and wcaG genes that are expressed under the control of the lac promoter.

12. The microorganism according to claim 11, wherein additional copies of the genes involved in the GDP-fucose biosynthetic pathway are integrated in the genome of the microorganism under control of a glp promoter.

13. The microorganism according to claim 12, wherein the polypeptide having an α1,3/4-fucosyl transferase activity is a protein that comprises the amino acid sequence set forth in SEQ ID No. 7 and its coding nucleic acid sequence is integrated in the genome of the microorganism in a single copy.

14. A method for producing LNFP-V in a recombinant microorganism, the method comprising:
a) providing a genetically modified microorganism according to claim 1,
b) culturing said microorganism in the presence of lactose, and
c) separating LNFP-V from said microorganism, from the culture medium or from both.

15. A protein that comprises the amino acid sequence set forth in SEQ ID No. 7.

16.-20. (canceled)

21. The microorganism according to claim 1, wherein the polypeptide having an α1,3/4-fucosyl transferase activity is selected from the group consisting of:
a protein that consists of the amino acid sequence set forth in SEQ ID No. 1,
a protein that consists of the amino acid sequence set forth in SEQ ID No. 3,
a protein that consists of the amino acid sequence set forth in SEQ ID No. 4, and
a protein that consists of the amino acid sequence set forth in SEQ ID No. 7.

22. The microorganism according to claim 1, wherein the polypeptide having an α1,3/4-fucosyl transferase activity is a protein that consists of the amino acid sequence set forth in SEQ ID No. 7.

23. The microorganism according to claim 10, wherein each heterologous nucleic acid sequence is integrated in a site of an operon related to a sugar metabolism

24. The microorganism according to claim 12, wherein the glp promoter is glpF or its variant according to SEQ ID No. 5.

25. The microorganism according to claim 13, wherein the polypeptide having an α1,3/4-fucosyl transferase activity is a protein that consists of the amino acid sequence set forth in SEQ ID No. 7


SEQUENCE LISTING (7 sequences)

SEQ ID NO: 1 - protein, 441 aa, Artificial Sequence
  Ma et al. J. Biol. Chem. 281, 6385 (2006)
MFQPLLDAYVESASIEKMASKSPPPLKIAVANWWGDEEIKEFKNSVLYFILSQRYTITLHQNPNEFSDLVFGNPLGSARKILSYQNAKRVFYTGENESPNFNLFDYAIGFDELDFNDRYLRMPLYYDRLHHKAESVNDTTAPYKLKDNSLYALKKPSHCFKEKHPNLCAVVNDESDPLKRGFASFVASNPNAPIRNAFYDALNSIEPVTGGGSVRNTLGYNVKNKNEFLSQYKFNLCFENTQGYGYVTEKIIDAYFSHTIPIYWGSPSVAKDFNPKSFVNVHDFKNFDEAIDYIKYLHTHKNAYLDMLYENPLNTLDGKAYFYQNLSFKKILAFFKTILENDTIYHDNPFIFCRDLNEPLVTIDDLRVNYDDLRVNYDDLRINYDDLRVNYDDLRINYDDLRVNYDDLRVNYDDLRINYDDLRVNYDDLRVNYERLLSKAT

SEQ ID NO: 2 - protein, 478 aa, Helicobacter pylori
  GenBank ID AAB81031.1
MFQPLLDAYVESASIEKMASKSPPPLKIAVANWWGDEEIKEFKNSVLYFILSQRYTITLHQNPNEFSDLVFGNPLGSARKILSYQNAKRVFYTGENESPNFNLFDYAIGFDELDFNDRYLRMPLYYDRLHHKAESVNDTTAPYKLKDNSLYALKKPSHCFKEKHPNLCAVVNDESDPLKRGFASFVASNPNAPIRNAFYDALNSIEPVTGGGSVRNTLGYNVKNKNEFLSQYKFNLCFENTQGYGYVTEKIIDAYFSHTIPIYWGSPSVAKDFNPKSFVNVHDFKNFDEAIDYIKYLHTHKNAYLDMLYENPLNTLDGKAYFYQNLSFKKILAFFKTILENDTIYHDNPFIFCRDLNEPLVTIDDLRVNYDDLRVNYDDLRINYDDLRVNYDDLRINYDDLRVNYDDLRVNYDDLRINYDDLRVNYDDLRVNYERLLSKATPLLELSQNTTSKIYRKAYQKSLPLLRAIRRWVKKLGL

SEQ ID NO: 3 - protein, 425 aa, Helicobacter pylori
  GenBank ID NP_207177.1
MFQPLLDAFIESASIEKMASKSPPPPLKIAVANWWGDEEIKEFKKSVLYFILSQRYAITLHQNPNEFSDLVFSNPLGAARKILSYQNTKRVFYTGENESPNFNLFDYAIGFDELDFNDRYLRMPLYYAHLHYKAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHKFSTSFMWEYDLHKPLVSIDDLRVNYDDLRVNYDRLLQNASPLLELSQNTTFKIYRKAYQKSLPLLRAVRKLVKKLGL

SEQ ID NO: 4 - protein, 425 aa, Artificial Sequence
  Choi et al. Biotechnol. Bioengin. 113, 1666 (2016)
MFQPLLDAFIESASIEKMASKSPPPPLKIAVANWWGDEEIKEFKKSVLYFILSQRYAITLHQNPNEFSDLVFSNPLGAARKILSYQNTKRVFYTGENESPNFNLFDYAIGFDELDFNDRYLRMPLYYNELHYKAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHKFSTSFMWEYDLHKPLVSIDDLRVNYDDLRVNYDRLLQNASPLLELSQNTTFKIYRKAYQKSLPLLRAVRKLVKKLGL

SEQ ID NO: 5 - DNA, 300 bp, Artificial Sequence
  PglpF variant
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 6 - protein, 439 aa, Artificial Sequence
  GalTK
MISVYIISLKESQRRLDTEKLVLESNEKFKGRCVFQIFDAISPKHEDFEKFVQELYDSSSLLKSDWFHSDYCYQELLPQEFGCYLSHYLLWKECVKLNQPVVILEDDVALESNFMQALEDCLKSPFDFVRLYGHYWGGHKTNLCALPVYTETEEAEASIEKTPIENYEVTSPPPPNPTRDTQQDFITETQQDPKELSEPCKIAPQKISFNQVVFKKIKRKLNRFIGSILARTEVYKNIVAKYDDLTTKYDDLTTKYDDLTTKYDDLTTKYDDLNKNIAEKYDELMGKYESLLAKEVNIKETFWESRADSEKEALFLDHFYLTSVYVATTAGYYLTPKGAKTFIEATERFKIIEPVDMFINNPTYHDIANFTYVPCPVSLNKHAFNSTIQNAKKPDISLKPPKKSYFDNLFYHKFNARKCLKAFNKYSKQYAPLKTPKEV

SEQ ID NO: 7 - protein, 425 aa, Artificial Sequence
  FutA mutant
MFQPLLDAFIESASIEKMASKSPPPPLKIAVANWWGDEEIKEFKKSVLYFILSQRYAITLHQNPNEFSDLVFSNPLGAARKILSYQNTKRVFYTGENESPNFNLFDYAIGFDELDFNDRYLRMPLYYNELHYKAELVNDTTAPYKLKGNSLYALKKPSHHFKENHPNLCAVVNDESDLLKRGFASFVASNANAPMRNAFYDALNSIEPVTGGGSVRNTLGCKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHKFSTSFMWEYDLHKPLVSIDDLRVNYDDLRVNYDRLLQNASPLLELSQNTTFKIYRKAYQKSLPLLRAVRKLVKKLGL